(19) United States
(12) Patent Application Publication
(10) Pub. No.: US 2023/0098714 A1
(43) Pub. Date: Mar. 30, 2023

(54) MICRORNA COMPOSITIONS AND METHODS OF USE

(71) Applicant: New York R&D Center for Translational Medicine and Therapeutics, Inc., New Rochelle, NY (US)

(72) Inventor: Hui B. SUN, New Rochelle, NY (US)

(21) Appl. No.: 17/882,009

(22) Filed: Aug. 5, 2022

Related U.S. Application Data
(60) Provisional application No. 63/230,449, filed on Aug. 6, 2021.

(51) Int. Cl. C12N 15/113; A61P 19/04

(52) U.S. Cl. CPC C12N 15/113; A61P 19/04; C12N 2310/141; C12N 2310/315; C12N 2310/321; C12N 2310/323; C12N 2310/3515

(57) ABSTRACT

Provided herein are compositions and methods of use for mimicking miRNA activity. For example, these compositions can be effective in modulating expression of pro-inflammatory mediators, such as NLRP3. The compositions can be useful for treating musculoskeletal or connective tissue repair or degenerative conditions, for example, tendinopathy and tendon injury.

### CROSS-REFERENCE

This application claims the benefit of U.S. Provisional Application No. 63/230,449, filed on Aug. 6, 2021, which is incorporated herein by reference.

### STATEMENT REGARDING FEDERALLY SPONSORED RESEARCH OR DEVELOPMENT

The invention was made with the support of the United States government under the Small Business Innovation Research (SBIR) Award #5R44AG056246-03 by the National Institutes of Health. The government may have certain rights in the invention.

### SEQUENCE LISTING

The instant application contains a Sequence Listing which has been submitted electronically in XML file format and is hereby incorporated by reference in its entirety. Said XML copy, created on Sep. 15, 2022, is named 53126-703_201_SL.xml and is 4,586 bytes in size.

### BACKGROUND

MicroRNAs (miRNAs) are small, single-stranded, non-coding RNA molecules that participate in RNA silencing and post-transcriptional regulation of gene expression. miRNAs can be used as biomarkers for disease and pharmaceutical therapeutics for medical intervention.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, the present disclosure provides a pharmaceutical composition comprising in a unit dosage form: a therapeutically-effective amount of a compound that increases activity of miRNA-221-5p; and a pharmaceutically-acceptable carrier.

In some embodiments, the present disclosure provides a method of treating a connective tissue condition in a subject in need thereof, the method comprising administering to the subject a therapeutically-effective amount of a compound that increases activity of miRNA-221-5p.

In some embodiments, the present disclosure provides a method of treating a musculoskeletal condition in a subject in need thereof, the method comprising administering to the subject a therapeutically-effective amount of a compound that increases activity of miRNA-221-5p.

### BRIEF DESCRIPTION OF THE DRAWINGS

FIG. 1, Panel A shows a sequence alignment of rat and human miR-221-5p. FIG. 1, Panel B illustrates that the sequence miR-221-5p is complementary with rat NLRP3 mRNA.

FIG. 2 shows PCR analysis data showing that miR-221-5p suppresses mRNA expression of pro-inflammatory mediators and downstream factors including genes involved in the synthesis and degradation of ECM: NLRP3, IL-18, MCP1, P65, IL-17A, TNFα, Col3a1, MMP1, and HIF-1α in rat tenocytes in vitro. Rat tenocytes cultured in vitro were treated with a miR-221-5p mimic (miR-221-5p) versus a scramble miRNA (Scr).

FIG. 3, Panel A shows a H&E analysis of rat calcaneal tendinopathic tendons treated with miR-Scr versus miR-221-5p, compared to non-tendinopathic sham control. Panel B shows Movin Scores of rat calcaneal tendinopathic tendons treated with miR-Scr versus miR-221-5p, compared to non-tendinopathic sham control. Panel C shows characterization of cleaved type I collagen in rat calcaneal tendinopathic tendons treated with miR-Scr versus miR-221-5p, compared to non-tendinopathic sham control. Panel D shows cleaved type I collagen levels in rat calcaneal tendinopathic tendons treated with miR-Scr versus miR-221-5p, compared to non-tendinopathic sham control. Panel E shows type I collagen (Col I) expression in rat calcaneal tendinopathic tendons treated with miR-Scr versus miR-221-5p, compared to non-tendinopathic sham control. Panel F shows type III collagen (Col III) expression in rat calcaneal tendinopathic tendons treated with miR-Scr versus miR-221-5p, compared to non-tendinopathic sham control. Panel G shows NLRP3 expression in rat calcaneal tendinopathic tendons treated with miR-Scr versus miR-221-5p, compared to non-tendinopathic sham control. Panel H shows IL-1β expression in rat calcaneal tendinopathic tendons treated with miR-Scr versus miR-221-5p, compared to non-tendinopathic sham control. Panel I shows TNFα expression in rat calcaneal tendons treated with miR-Scr versus miR-221-5p, compared to non-tendinopathic sham control. Panel J shows Von Frey assay results of rat calcaneal tendinopathic tendons treated with miR-Scr versus miR-221-5p, compared to non-tendinopathic sham control. S, sham; Scr, miR-Scr; miR, miR-221-5p.

FIG. 4, Panel A shows a schematic of chemically-modified antisense strands of miR-221-5p mimics. Panels B and C show representative images and quantification of the immunofluorescent intensity of cells transfected with miR-221-5p mimics at 24 and 72 hours after transfection in human tenocytes (87-year-old male) in the presence of IL-1β; FAM in green color; DAPI in blue color; n=4-5 images per experimental group. Panels D and E show representative images and quantification of NLRP3 expression (red) in the transfected tenocytes in vitro compared to DAPI (blue). Panels F and G show quantification of FAM and NLRP3 immunofluorescence staining in tendinopathic tissue in vivo.

### DETAILED DESCRIPTION

Provided herein are compositions and methods of use in treating musculoskeletal or connective tissue conditions, for example, tissue repair or degenerative conditions such as tendinopathy and tendon injury. For example, compositions described herein can be used for the treatment of tendinopathy and enhancing healing from a tendon injury. Such compositions can mitigate disease severity and relieve tendinopathy-related pain and discomfort. The compositions described herein include one or more compounds that enhance and/or mimic the activity of miRNA-221-5p in a subject.

### Tendinopathy and Tendon Injury

Tendinopathy is a highly-prevalent tendon disorder. Tendinopathy is chronic tendon degeneration. In most cases, tendinopathy is caused by tendon overuse and/or overload, leading to microscopic collagen fiber failure and a failed healing response, often resulting in tendon rupture. While tendinopathy can affect any tendon, the most common incidents occur on the rotator cuff tendons, the Achilles tendon, tennis elbow, and golfer's elbow.

Tendonitis is the inflammation of a tendon. Like tendinopathy, tendonitis can occur when a tendon is overused or injured, for example, during sport or physical activities. Tendonitis is often linked to an acute injury with inflammation. Tendonitis can affect tendons in the elbow, wrist, finger, thigh, and other parts of the body. Non-limiting examples of sites of tendon injury or tendon degeneration include the rotator cuff, the Achilles, tennis elbow (tissue connecting forearm muscle to elbow), golfer's elbow (inner side of elbow), jumper's knee (also known as patellar tendon; tissue connecting kneecap to shinbone), swimmer's shoulder (tissue rubbing against shoulder blade), the neck, the forearm, the shoulder, the pelvis, the hip, the hamstring, the knee, the ankle, and the foot.

Treatment strategies for tendon injuries and degenerative conditions include administration of non-steroidal anti-inflammatory drugs (NSAIDs), corticosteroid injections, platelet-rich plasma (PRP) injections, and physical therapy. However, many of these strategies tend to be limited to targeting only the symptomatology instead of intervening the disease pathology. In addition, prolonged use of NSAIDs can elevate the risk of upset stomach and gastrointestinal bleeding, while corticosteroids can deleteriously affect tendon tissue. Alternative therapeutics, such as compositions described herein, can be useful for treating or managing tendon dysfunction.

miRNAs

miRNAs are small, non-coding RNAs that regulate gene expression through repression of translation, either through inhibition of translation or induction of mRNA degradation. For example, miRNAs can exert regulatory function through complementary base pairing to the 3′-untranslated regions (3′-UTRs) of protein-coding mRNAs. miRNAs can be derived from primary RNA transcripts (pri-miRNA) synthesized by RNA pol II, which can be several thousand nucleotides in length. A single pri-miRNA transcript can give rise to more than one active miRNA. In the nucleus, the type III RNAse enzyme Drosha processes the pri-miRNA transcript into a precursor miRNA (pre-miRNA). The pre-miRNA consists of a stem-loop or hairpin structure and is generally around 70 to 100 nucleotides in length.

The pre-miRNA is then transported to the cytoplasm, where the pre-miRNA is further processed by the RNAse Dicer, which removes the loop to yield a mature double-stranded miRNA molecule having an active “guide” or “sense” strand (generally 15 to 25 nucleotides in length) hybridized to a fully or partially complementary “passenger” or “antisense” strand. The mature, double-stranded miRNA is then incorporated into the RNA-induced silencing complex (RISC), where the guide strand hybridizes to a binding site in the target mRNA. The guide strand is not always completely complementary to the target binding site. However, a region of the guide strand designated as the “seed” sequence is often fully complementary to the corresponding sequence of the target binding site. The seed sequence is generally 2 to 8 nucleotides in length and located at or near (within 1-2 nucleotides of) the 5′-end of the guide strand.

Single, unpaired guide strands are also capable of being incorporated into RISC. Modifications to the passenger strand (e.g., to the sugars, the bases, or the backbone structure), which impede incorporation of the passenger strand into RISC, can also increase efficiency of target inhibition by a double-stranded miRNA.

A guide strand can be single stranded or can be hybridized with a second “passenger” RNA strand. The guide strand and passenger strand run anti-parallel to one another in the hybridized complex and are double-stranded miRNA. The guide strand, when present in isolation, is single-stranded miRNA. The passenger strand and the guide strand can contain a number of mismatches, resulting in bulges (i.e., an unpaired nucleotide or plurality of consecutive unpaired nucleotides within one strand) or internal loops (opposed unpaired nucleotides in both strands). One or more nucleotides at the termini are sometimes unpaired. Alternatively, the passenger strand can be 100% complementary to the seed sequence of the guide strand.

In some embodiments, provided herein are miRNA compositions that enhance and/or mimic the activity of miRNA-221-5p. Similar to PRP treatment, miRNA therapeutics do not involve the use of live cells during product production, storage, transportation, and administration. miRNAs can be derived from various sources, such as isolation from microvesicles or synthetic production. miRNA therapeutics can also be cryopreserved for several years. Therefore, one-time production of a miRNA composition can be used for multiple treatments. As miRNAs are oligonucleotide chains of limited length, miRNA compositions described herein can be synthesized using oligonucleotide engineering technologies.

### Modulation of the NLRP3 Inflammasome

NLR family pyrin domain containing 3 (NLRP3) is a protein encoded by the NLRP3 gene in humans. NLRP3 is expressed predominantly in macrophages and as a component of the inflammasome. NLRP3 functions as a pattern recognition receptor (PRR) that recognizes pathogen-associated molecular patterns (PAMPs). For example, NLRP3 can detect products of damaged cells such as extracellular ATP and crystalline uric acid. Activated NLRP3 triggers an immune response. Mutations in the NLRP3 gene are associated with a number of organ specific autoimmune diseases. The NLRP3 inflammasome can be a potential drug target for diseases underpinned by inflammation.

miRNA-221-5p, is a small, noncoding RNA sequence with 22 bases. Homo sapiens (human) miR-221-5p (hsa-miR-221-5p) has the sequence: ACCUGGCAUACAAUGUAGAUUU (SEQ ID NO: 2). The hsa-miR-221-5p sequence is a product of miR-221, hsa-miR-221, miR-221-5p, MIR221, hsa-miR-221-5p genes. miRNA-221-5p can regulate the activation of the NLRP3 inflammasome by inhibiting NLRP3 expression through binding to NLRP3 3′-UTR. miRNA-221-5p can further suppress pro-inflammatory mediators that contribute to the pathogenesis of tendinopathy. These mediators can include genes of type I collagen (collagen I) and type III collagen (collagen III), and other genes involved in the synthesis and degradation of the extracellular matrix (ECM). These pro-inflammatory mediators include inflammatory cytokines such as IL-1β and TNFα. Secretion of IL-1β can occur in response to molecular motifs carried by PAMPs. Elevated IL-1β levels can exacerbate damage during chronic disease and acute tissue injury.

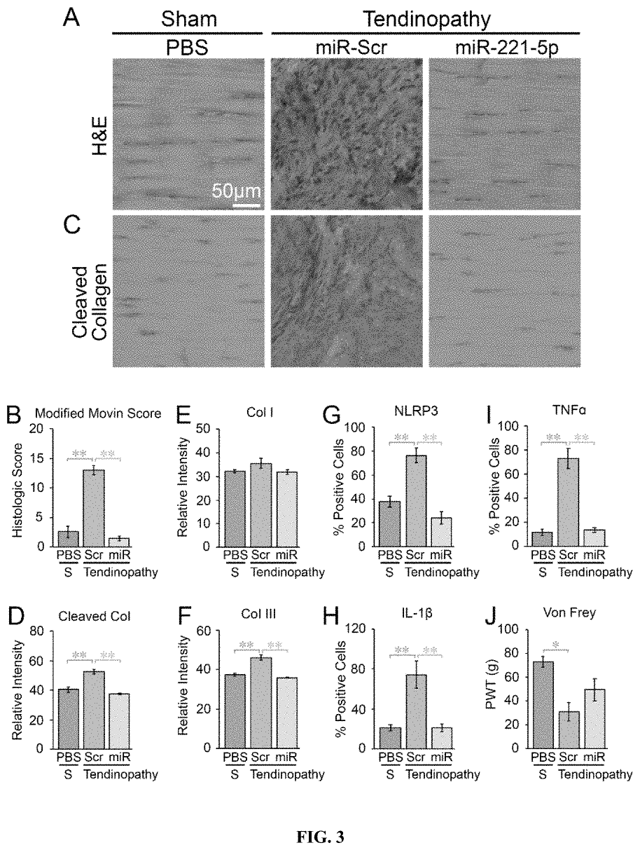

Local administration of miR-221-5p can exert a therapeutic effect in tendinopathy by restoring tendon histomorphology and expression of proinflammatory mediators and matrix proteins, in addition to mitigation of pain-related behavior (see FIG. 3).

As such, miRNA-221-5p or a mimic thereof can be used to mitigate tendinopathy by mediating mechanisms of tissue repair, such as suppression of NLRP3, a key regulator of tendinopathy, and a spectrum of tendinopathy-related pro-inflammatory mediators that contribute to the pathogenesis of tendinopathy. Treatment of tendinopathic tendons using compositions described herein can lead to restoration of the degraded tissue microstructure and histology, increased expression of markers for tissue repair, and improved overall joint mobility, while also relieving symptoms of tendinopathy-related discomfort and pain.

### Pharmaceutical Compositions

A pharmaceutical composition disclosed herein can provide a therapeutically-effective amount of a compound that mimics, increases, or enhances the biological activity of miRNA-221-5p. Such a compound is referred to herein as a miRNA-221-5p mimic. A miRNA-221-5p mimic disclosed herein has the same or substantially the same biological activity of naturally-occurring miRNA-221-5p in vitro or in vivo, e.g., in a subject. In some embodiments, miRNA-221-5p mimic disclosed herein has enhanced biological activity of naturally-occurring miRNA-221-5p in vitro or in vivo. A miRNA-221-5p mimic disclosed herein includes a NLRP3 binding agent that causes inhibition or reduction in the activity of NLRP3. A miRNA-221-5p mimic disclosed herein can reduce the abundance of a mRNA encoding NLRP3 or the NLRP3 protein, e.g., the active form of NLRP3, thereby reducing the biological activity of NLRP3. In some embodiments, a miRNA-221-5p mimic disclosed herein can inhibit or reduce a post-translational modification of NLRP3.

Non-limiting examples of miRNA-221-5p mimics include small organic or inorganic molecules, antibodies, darpins (i.e., a genetically engineered antibody mimetic protein), peptides, aptamers (an oligonucleotide or peptide molecule that can bind to a specific target molecule), adnectins (an antibody mimic), peptibodies (a molecule comprising an antibody Fc domain attached to at least one peptide), proteins, and nucleic acids (such as RNA or DNA oligonucleotides, e.g., miRNA). In some embodiments, a miRNA-221-5p mimic is a miRNA.

A miRNA-221-5p mimic disclosed herein can be covalently or non-covalently conjugated to another moiety. This additional moiety can, for example, enhance stability, inhibit degradation, increase half-life, increase absorption, reduce toxicity, reduce immunogenicity, and/or increase biological activity of the miRNA-221-5p or mimic thereof. The moiety can be conjugated to the 5′-end or the 3′-end of the miRNA. The moiety can be incorporated into a compound by chemical modification. Non-limiting examples of the additional moiety include Fc domains of immunoglobulins, polymers such as polyethylene glycol (PEG), polylysine, dextran, lipids, cholesterol groups (such as steroids), carbohydrates, dendrimers, oligosaccharides, and peptides. Addition of a cholesterol group to a compound can facilitate cellular uptake and increase pharmacokinetic properties of the compound.

Chemical modifications can be used in drug development to enhance the stability of a drug and delivery to the target. A compound disclosed herein can be chemically modified to enhance uptake and stability of the compound. A compound described herein can include a combination of one or more chemical modifications described herein.

For example, modification of RNA at the 2′-position improves biostability of the original RNA by increasing resistance against nucleolytic attack. In some embodiments, a compound herein is an RNA comprising a 2′-modification, e.g., 2′-O-methyl-RNA, 2′-O-(2-methoxyethyl)-RNA, or 2′-O-flouro-RNA. In some embodiments, a compound disclosed herein is a 2′-O-methylated RNA.

In some embodiments, a compound of the disclosure is conjugated to a serum lipoprotein, such as cholesterol, vitamin E, palmitate, tocopherol, or a combination thereof.

In some embodiments, a compound of the disclosure contains a ribose modification, such as a 2′-O-methyl, 2′-fluoro, 2′-fluoroarabino, 2′-O-methoxyethyl, 4′-thioribonucleoside, 4′-C-aminomethyl-2′-O-methyl, deoxyribonucleotide, cyclohexenyl, hexitol modification, or a combination thereof.

In some embodiments, a compound of the disclosure is a locked nucleic acid. A locked nucleic acid is in which the ribose moiety is modified with an additional bridge connecting the 2′-oxygen and 4′-carbon. In some embodiments, a compound of the disclosure is an unlocked nucleic acid. An unlocked nucleic acid is an acyclic RNA analogue in which the bond between the C2′ and C3′ atoms of the ribose ring is cleaved.

In some embodiments, a compound of the disclosure contains one or more phosphate backbone modifications, such as a phosphorothioate, dimethylethylenediamine, tert-butyl-S-acyl thioethyl (tBu-SATE), boranophosphate, amide linker, or 5′-phosphate modification, e.g., 5′-C-methyl, 5′-(E)-vinylphosphonate (5′-E-VP), or 5′-methylene phosphonate modification.

In some embodiments, a compound herein is a chemically-modified miRNA. The chemical modification can be on the guide strand (sense strand), the passenger strand (antisense strand), or both.

In some embodiments, a compound herein is an oligonucleotide comprising one or more phosphorothioate (PS) backbone modifications. This modification renders internucleotide linkages resistant to nuclease degradation. Phosphorothioate bonds can be introduced between the nucleotides at the 5′- or 3′-end of an oligonucleotide to inhibit exonuclease degradation.

In some embodiments, a pharmaceutical composition disclosed herein provides a therapeutically-effective amount of a modulator of NLRP3, for example, an inhibitor of NLRP3. A pharmaceutical composition disclosed herein can provide a therapeutically-effective amount of an inhibitor of pro-inflammatory mediators including, for example, IL-1β, IL-6, IL-8, IL-10, and TNFα.

In some embodiments, a pharmaceutical composition disclosed herein provide a therapeutically-effective amount of an oligonucleotide or a derivative thereof. In some embodiments, a pharmaceutical composition disclosed herein provide a therapeutically-effective amount of a miRNA or a mimic thereof, a precursor of a miRNA or a mimic thereof, a nucleic acid encoding a miRNA or a mimic thereof, or a nucleic acid encoding a precursor of a miRNA or a mimic thereof. In some embodiments, the miRNA is miRNA-221-5p. Non-limiting examples of precursors of miR-221-5p include pre-mir-221-5p and pri-mir-221-5p, and fragments and variants thereof that can be processed to mature miR-221-5p.

A miRNA or a mimic thereof can include modifications to include, for example, one or more modified sugar residues, one or more modified internucleoside linkages, one or more modified bases, a targeting moiety, a membrane transit moiety. A miRNA or a mimic thereof can be associated with (i.e., conjugated with, complexed with, or encapsulated by) a carrier. The carrier can be a pharmaceutically-acceptable lipid or polymer. The carrier molecule can include a targeting agent capable of binding to a surface of a target cell.

Nucleic acids encoding miR-221-5p or a mimic thereof, or a precursor of miR-221-5p or a mimic thereof, can be administered to a subject to be expressed in a cell of the subject, e.g., a tendon cell (tenocyte). Nucleic acids can be associated with (i.e., conjugated with, complexed with, or encapsulated by) a carrier. The carrier can be a pharmaceutically-acceptable lipid or polymer. The carrier molecule can include a targeting agent capable of binding to a surface of a target cell. Nucleic acids can be delivered administered to a subject via a viral vector. Non-limiting examples of viral vectors include an adenovirus vector, an adeno-associated virus (AAV) vector, a retrovirus vector, a lentiviral vector, or a herpesvirus vector.

A nucleic acid encoding a miRNA-221-5p or a mimic thereof, such as an antibody binding NLRP3, is delivered by a suitable method, for example, a recombinant viral vector, to a subject in need thereof. The nucleic acid can include, for example, exons encoding miRNA-221-5p or a mimic thereof, an intron, an enhancer region, a promoter region, and a transcription terminator region.

Delivery of a nucleic acid to a cell, referred to as transfection, can be accomplished by a number of methods. Viral nucleic acid delivery methods use recombinant viruses for nucleic acid transfer. Non-viral nucleic acid delivery can comprise injecting naked DNA or RNA, use of carriers including lipid carriers, polymer carriers, chemical carriers, and biological carriers such as biologic membranes, bacteria, and virus-like particles, and physical/mechanical approaches. A combination of viral and non-viral nucleic acid delivery methods can be used for efficient gene therapy.

Non-viral nucleic acid transfer can include injection of naked nucleic acid, for example, nucleic acid that is not protected and/or devoid of a carrier. In vivo, naked nucleic acid can be subject to rapid degradation, low transfection levels, and poor tissue-targeting ability. Hydrodynamic injection methods can increase the targeting ability of naked nucleic acids.

Non-viral nucleic acid delivery systems can include chemical carriers. These systems can include lipoplexes, polyplexes, dendrimers, and inorganic nanoparticles. A lipoplex is a complex of a lipid and a nucleic-acid that protects the nucleic acid from degradation and facilitates entry into cells. Lipoplexes can be prepared from neutral, anionic, and/or cationic lipids. Preparation of lipoplexes with cationic lipids can facilitate encapsulation of negatively charged nucleic acids. Lipoplexes with a net positive charge can interact more efficiently with a negatively charged cell membrane. Preparation of lipoplexes with a slight excess of positive charges can confer higher transfection efficiency. Lipoplexes can enter cells by endocytosis. Once inside the cell, lipoplexes can release the nucleic acid contents into the cytoplasm. A polyplex is a complex of a polymer and a nucleic acid. Most polyplexes are prepared from cationic polymers that facilitate assembly by ionic interactions between nucleic acids and polymers. Uptake of polyplexes into cells can occur by endocytosis. Inside the cells, polyplexes require co-transfected endosomal rupture agents such as inactivated adenovirus, for the release of the polyplex particle from the endocytic vesicle. Examples of polymeric carriers include polyethyleneimine, chitosan, poly(beta-amino esters), and polyphosphoramidate. Polyplexes show low toxicity, high loading capacity, and ease of fabrication. A dendrimer is a highly branched molecule. Dendrimers can be constructed to have a positively-charged surface and/or carry functional groups that aid temporary association of the dendrimer with nucleic acids. These dendrimer-nucleic acid complexes can be used for gene therapy. The dendrimer-nucleic acid complex can enter the cell by endocytosis. Nanoparticles prepared from inorganic material can be used for nucleic acid delivery. Examples of inorganic material can include gold, silica/silicate, silver, iron oxide, and calcium phosphate. Inorganic nanoparticles with a size of less than 100 nm can be used to encapsulate nucleic acids efficiently. The nanoparticles can be taken up by the cell via endocytosis. Inside the cell, the nucleic acid can be released from the endosome without degradation. Nanoparticles based on quantum dots can be prepared and offer the use of a stable fluorescence marker coupled with gene therapy. Organically modified silica or silicate can be used to target nucleic acids to specific cells in an organism.

Non-viral nucleic acid delivery systems can include biological methods including bactofection, biological liposomes, and virus-like particles (VLPs). Bactofection method comprises using attenuated bacteria to deliver nucleic acids to a cell. Biological liposomes, such as erythrocyte ghosts and secretion exosomes, are derived from the subject receiving gene therapy to avoid an immune response. Virus-like particles (VLP) or empty viral particles are produced by transfecting cells with only the structural genes of a virus and harvesting the empty particles. The empty particles are loaded with nucleic acids to be transfected for gene therapy.

Delivery of nucleic acids described herein can also be enhanced by physical methods. Examples of physical methods include electroporation, gene gun, sonoporation, and magnetofection. The electroporation method uses short high-voltage pulses to transfer nucleic acid across the cell membrane. These pulses can lead to formation of temporary pores in the cell membrane, thereby allowing nucleic acid to enter the cell. Electroporation can be efficient for a broad range of cells. Electron-avalanche transfection is a type of electroporation method that uses very short, for example, microsecond, pulses of high-voltage plasma discharge for increasing efficiency of nucleic acid delivery. The gene gun method utilizes nucleic acid-coated gold particles that are shot into the cell using high-pressure gas. Force generated by the gene gun allows penetration of nucleic acid into the cells, while the gold is left behind on a stopping disk. The sonoporation method uses ultrasonic frequencies to modify permeability of cell membrane. Change in permeability allows uptake of nucleic acid into cells. The magnetofection method uses a magnetic field to enhance nucleic acid uptake. In this method, nucleic acid is complexed with magnetic particles. A magnetic field is used to concentrate the nucleic acid complex and bring them in contact with cells.

Viral nucleic acid delivery systems use recombinant viruses to deliver nucleic acids for gene therapy. Non-limiting examples of viruses that can be used to deliver nucleic acids described herein include retrovirus, adenovirus, herpes simplex virus, adeno-associated virus, vesicular stomatitis virus, reovirus, vaccinia, pox virus, and measles virus.

Retroviral vectors can be used in the disclosure. Retrovirus is an enveloped virus that contains a single-stranded RNA genome. Retroviruses can integrate inside a host cell via reverse transcription. Retroviruses can enter a host cell by binding to specific membrane-bound receptors. Inside the host cell cytoplasm, retroviral reverse transcriptase generates double-stranded DNA from the viral RNA genome template. Retroviral enzyme integrase incorporates the new viral DNA into host cell genome, where the viral DNA is transcribed and translated along with host cell genes. Retroviral gene therapy vectors can be used for chromosomal integration of the transferred vector genomes, thereby leading to stable genetic modification of treated cells. Non-limiting examples of retroviral vectors include Moloney murine leukemia viral (MMLV) vectors, HIV-based viral vectors, gammaretroviral vectors, C-type retroviral vectors, and lentiviral vectors. Lentivirus is a subclass of retrovirus. While some retroviruses can infect only dividing cells, lentiviruses can infect and integrate into the genome of actively dividing cells and non-dividing cells.

Adenovirus-based vectors can be used in the disclosure. Adenovirus is a non-enveloped virus with a linear double-stranded genome. Adenoviruses can enter host cells using interactions between viral surface proteins and host cell receptors that lead to endocytosis of the adenovirus particle. Once inside the host cell cytoplasm, the adenovirus particle can be released by the degradation of the endosome. Using cellular microtubules, the adenovirus particle can gain entry into the host cell nucleus, where adenoviral DNA can be released. Inside the host cell nucleus, the adenoviral DNA can be transcribed and translated. Adenoviral DNA is not integrated into the host cell genome. Adenoviral DNA is not replicated during host cell division. Gene therapy using adenoviral vectors can require multiple administrations if the host cell population is replicating.

Herpes simplex virus (HSV)-based vectors can be used in the disclosure. HSV is an enveloped virus with a linear double-stranded DNA genome. Interactions between surface proteins on the host cell and HSV lead to pore formation in the host cell membrane. These pores allow HSV to enter the host cell cytoplasm. Inside the host cell, HSV uses the nuclear entry pore to enter the host cell nucleus where HSV DNA is released. HSV can persist in host cells in a state of latency. Herpes simplex virus 1 and 2 (HSV-1 and HSV-2), also known as human herpes virus 1 and 2 (HHV-1 and HHV-2), are members of the herpes virus family.

Alphavirus-based vectors can be used to deliver nucleic acids described herein. Examples of alphavirus-based vectors include vectors derived from semliki forest virus and sindbis virus. Alphavirus-based vectors can provide high transgene expression and the ability to transduce a wide variety of cells. Alphavirus vectors can be modified to target specific tissues. Alphaviruses can persist in a latent state in host cells, thereby offering the advantage of long-term nucleic acid expression in the cell.

Pox/vaccinia-based vectors such as orthopox or avipox vectors can be used in the disclosure. Pox virus is a double stranded DNA virus that can infect diving and non-dividing cells. Pox viral genome can accommodate up to 25 kb transgenic sequence. Multiple genes can be delivered using a single vaccinia viral vector.

In one aspect, the present disclosure provides a recombinant virus, such as an adeno-associated virus (AAV), as a vector to deliver a nucleic acid encoding miRNA-221-5p or a mimic thereof to a subject in need thereof.

Adeno-associated virus (AAV) is a small, nonenveloped virus that belongs to the Parvoviridae family. AAV genome is a linear single-stranded DNA molecule of about 4,800 nucleotides. The AAV DNA comprises two inverted terminal repeats (ITRs) at both ends of the genome and two sets of open reading frames. The ITRs serve as origins of replication for the viral DNA and as integration elements. The open reading frames encode for the Rep (non-structural replication) and Cap (structural capsid) proteins. AAV can infect dividing cells and quiescent cells. AAV is common in the general population and can persist naturally in the host.

AAV can be engineered for use as a gene therapy vector by substituting the coding sequence for both AAV genes with a transgene (transferred nucleic acid) to be delivered to a cell. The substitution eliminates immunologic or toxic side effects due to expression of viral genes. The transgene can be placed between the two ITRs (145 bp) on the AAV DNA molecule. AAV-based vectors can transencapsidate the genome allowing large variations in vector biology and tropism.

When producing recombinant AAV (rAAV), the viral genes and/or adenovirus genes providing helper functions to AAV can be supplied in trans to allow for production of the rAAV particles. In this way, rAAV can be produced through a three-plasmid system, decreasing the probability of production of wild-type virus.

AAV vector of the present disclosure can be generated using any AAV serotype. Non-limiting examples of serotypes include AAV1, AAV2, AAV2.5, AAV3, AAV4, AAV5, AAV6, AAV7, AAV8, AAV9, AAV10, AAV11, AAV12, rh10, and hybrids thereof.

AAV vectors can be modified for immune evasion or to enhance therapeutic output. The modifications can include genetic manipulation of the viral capsid. Proteins in the viral capsid can be rationally designed. The viral capsid can be modified by introducing exogenous agents such as antibodies, copolymers, and cationic lipids to evade the immune system. AAV vectors can be engineered to enhance the targeting ability. Targeting peptides and/or ligands can be inserted onto the capsid surface to enhance transduction into specific tissue. Capsid proteins from more than one serotype of AAV can be combined to produce a mosaic AAV vector comprising a capsid particle with enhanced targeting ability of the AAV vector. Tissue-specific promoters can be added to the viral vector to express the transgene in desired tissue types.

AAV vector can be modified to be self-complementary. A self-complementary AAV vector can comprise both strands of the viral DNA, thereby alleviating the requirement for host-cell second-strand DNA synthesis. The use of self-complementary AAV vectors can promote efficient transfer of nucleic acids into host genome.

A pseudotyped virus can be used for the delivery of nucleic acids. Pseudotyping involves substitution of endogenous envelope proteins of the virus by envelope proteins from other viruses or chimeric proteins. The foreign envelope proteins can confer a change in host tropism or alter stability of the virus. An example of a pseudotyped virus useful for gene therapy includes vesicular stomatitis virus G-pseudotyped lentivirus (VSV G-pseudotyped lentivirus) that is produced by coating the lentivirus with the envelope G-protein from Vesicular stomatitis virus. VSV G-pseudotyped lentivirus can transduce almost all mammalian cell types.

A hybrid vector having properties of two or more vectors can be used for nucleic acid delivery to a host cell. Hybrid vectors can be engineered to reduce toxicity or improve therapeutic transgene expression in target cells. Non-limiting examples of hybrid vectors include AAV/adenovirus hybrid vectors, AAV/phage hybrid vectors, and retrovirus/adenovirus hybrid vectors.

A viral vector can be replication-competent. A replication-competent vector contains all the genes necessary for replication, making the genome lengthier than replication-defective viral vectors. A viral vector can be replication-defective, wherein the coding region for the genes essential for replication and packaging are deleted or replaced with other genes. Replication-defective viruses can transduce host cells and transfer the genetic material, but do not replicate. A helper virus can be supplied to help a replication-defective virus replicate.

A viral vector can be derived from any source, for example, humans, non-human primates, dogs, fowl, mouse, cat, sheep, and pig.

The composition and methods of the disclosure provide for the delivery of a nucleic acid that encodes for a miRNA-221-5p or mimic thereof, or a NLRP3 suppressor to a subject in need thereof. The nucleic acid can be delivered by a viral vector, for example, an adeno-associated virus (AAV), adenovirus, retrovirus, herpes simplex virus, lentivirus, poxvirus, hemagglutinating virus of Japan-liposome (HVJ) complex, Moloney murine leukemia virus, or HIV-based virus. The nucleic acid can be delivered by a suitable non-viral method, for example, injection of naked nucleic acid, use of carriers such as lipid, polymer, biological or chemical carriers, or physical/mechanical approaches. The nucleic acid can be delivered by a combination of viral and non-viral methods.

Nucleic acids described herein can be generated using any method. The nucleic acids can be synthetic, recombinant, isolated, and/or purified. The nucleic acids can comprise, for example, a nucleic acid sequence that encodes for.

A vector described herein can comprise one or more nucleic acid sequences, each of which encodes a miRNA-221-5p or mimic thereof, or a NLRP3 suppressor.

A vector of the present disclosure can comprise one or more types of nucleic acids. The nucleic acids can include DNA or RNA. RNA nucleic acids can include a transcript of a gene of interest, for example, a miRNA-221-5p mimic, a NLRP3 suppressor, introns, untranslated regions, and termination sequences, or short interfering RNAs targeting NLRP3. DNA nucleic acids can include the gene of interest, promoter sequences, untranslated regions, and termination sequences. A combination of DNA and RNA can be used. The nucleic acids can be double-stranded or single-stranded. The nucleic acid can include non-natural or altered (modified) nucleotides.

A vector disclosed herein can comprise additional nucleic acid sequences including promoters, enhancers, repressors, insulators, polyadenylation signals (polyA), untranslated regions (UTRs), termination sequences, transcription terminators, internal ribosome entry sites (IRES), introns, origins of replication sequence, primer binding sites, att sites, encapsidation sites, polypurine tracts, long terminal repeats (LTRs), and linker sequences. The vector can be modified to target specific cells, for example, musculoskeletal cells, such as tenocytes.

Expression of a miRNA-221-5p or mimic thereof can be under the control of a regulatory sequence. The regulatory sequence can comprise a promoter. Promoters from any suitable source including virus, mammal, human, insect, plant, yeast, and bacteria, can be used. Tissue-specific promoters can be used. Promoters can be constitutive, inducible, or repressible. Promoters can be unidirectional (initiating transcription in one direction) or bi-directional (initiating transcription in either a 3′ or 5′ direction). Non-limiting examples of promoters include the T7 bacterial expression system, pBAD (araA) bacterial expression system, the cytomegalovirus (CMV) promoter, the SV40 promoter, the Rous sarcoma virus promoter, MMT promoter, EF-1 alpha promoter, UB6 promoter, chicken beta-actin promoter, CAG promoter, RPE65 promoter, opsin promoter, HIV-1 promoter, HIV-2 promoter, AAV promoter, adenovirus promoters such as from the E1A, E2A, or MLP region, cauliflower mosaic virus promoter, HSV-TK promoter, avian sarcoma virus promoter, MLV promoter, MMTV promoter, and rat insulin promoter. Inducible promoters can include, for example, the Tet system, the ecdysone inducible system, the T-REX™ system, LACSWITCH™ System, and the Cre-ERT tamoxifen inducible recombinase system.

Pre-miR-221-5p can refer to an RNA oligonucleotide consisting of a full-length mammalian pre-mir-221-5p sequence, or a fragment or variant thereof that comprises a mature miR-221-5p guide sequence connected by a loop sequence to a corresponding passenger sequence that is fully or partially complementary to the guide sequence. The oligonucleotide can form a stem-loop structure (or “hairpin”) in which the guide sequence and passenger sequence hybridize to one another.

Pri-miR-221-5p can refer to an RNA oligonucleotide consisting of a full-length mammalian pri-mir-221-5p sequence, or a fragment or variant thereof that comprises a pre-mir-221-5p sequence and is capable of being processed to a pre-mir-221-5p sequence by the double-stranded RNA-specific ribonuclease (RNAse III-type enzyme) Drosha.

A pharmaceutical composition can be a combination of any pharmaceutical compounds described herein with other chemical components, such as carriers, stabilizers, diluents, dispersing agents, suspending agents, thickening agents, solubilizing agents, excipients, buffers, preservatives, and surface active agents. For example, a pharmaceutical composition disclosed herein is suspended in a saline solution. Pharmaceutical formulations can also include one or more additional active ingredients such as antimicrobial agents, anti-inflammatory agents, or anesthetics.

The pharmaceutical composition facilitates administration of the compound to a subject. Pharmaceutical compositions can be administered by various forms and routes including, for example, injection, peri-tissue injection, intra-tissue injection, intratendinous injection, intraarticular, intramuscular, intravascular, subcutaneous, infusion, intravenous, enteral, oral, buccal, sublingual, rectal, parenteral, ocular, and topical administration. In some embodiments, a pharmaceutical composition disclosed herein can be administered via local injection into the pathological site of a subject. For example, a pharmaceutical composition disclosed herein can be administered via local injection into a tendon of a subject. In some embodiments, a pharmaceutical composition disclosed herein can be administered via a biocompatible substrate, e.g., an implant, scaffold, vesicle, or liposome.

Pharmaceutical compositions disclosed herein can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

Parenteral injections can be formulated for bolus injection or continuous infusion. Pharmaceutical compositions can be in a form suitable for parenteral injection as a sterile suspension, solution, or emulsion in oily or aqueous vehicles, and can contain formulatory agents, such as suspending, stabilizing, or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

Pharmaceutical compositions can be formulated using one or more physiologically-acceptable carriers comprising excipients and auxiliaries, which facilitate processing of the active compounds into preparations that can be used pharmaceutically. An excipient can fill a role as simple and direct as being an inert filler, or an excipient as used herein can be part of a pH stabilizing system or coating to ensure delivery of the ingredients safely to a target tissue. Formulations can be modified depending upon the route of administration chosen. Pharmaceutical compositions comprising a compound described herein can be manufactured, for example, by mixing, dissolving, granulating, dragee-making, levigating, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. The methods and pharmaceutical compositions described herein can include the use of crystalline forms (or polymorphs), and active metabolites of these compounds having the same type of activity.

A compound disclosed herein can be produced by various methods in any quantity. For example, a compound disclosed herein can be produced in an amount of about 1 microgram, about 1 milligram, about 1 gram, about 1 kilogram, or more. Non-limiting examples of production methods include in vitro transcription methods, polymerase chain transcription (PCT), recombinant overexpression (e.g., in E. coli, R. sulfidophilum, or other in vitro systems), transfer RNA (tRNA) scaffold methods, enzymatic methods, chemical methods, solid-phase oligonucleotide synthesis, solid-phase chemical synthesis, ribozyme cleavage methods, T4 ligation methods, position-selective labeling of RNA (PLOR), T7 RNA polymerase in vitro methods, T3 RNA polymerase in vitro methods, SP6 RNA polymerase in vitro methods, phosphoramidite chemistry, cell-free nucleic acid expression methods, or a combination thereof. Non-limiting examples of purification methods include precipitation and solvent extraction, ultracentrifugation, polyacrylamide gel electrophoresis (PAGE), liquid chromatography (e.g., reversed-phase ion-pairing HPLC (RP-IP-HPLC), ion-exchange HPLC (IE-HPLC), ion-exchange fast-performance liquid chromatography (IE-FPLC), affinity chromatography (e.g., systematic evolution of ligands by exponential enrichment (SELEX), and size-exclusion chromatography (SEC)), or a combination thereof. Purification methods can be used to achieve varying degrees of purity of a compound disclosed herein, e.g., at least 80% purity, at least 85% purity, at least 90% purity, at least 91% purity, at least 92% purity, at least 93% purity, at least 94% purity, at least 95% purity, at least 96% purity, at least 97% purity, at least 98% purity, at least 99% purity, or at least 99.99%. Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, nanoparticles, vesicles, microvesicles, or nanovesicles comprising a compound as disclosed herein. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the present disclosure include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

In some embodiments, a compound disclosed herein is present in a pharmaceutical composition or unit dosage form in an amount of from about 0.1 picomole (pmol) to about 100 pmol, about 0.1 nanomole (nmol) to about 100 nmol, about 0.1 micromole (μmol) to about 100 μmol, about 0.1 millimole (mmol) to about 100 mmol, about 0.1 mole (mol) to about 100 mol; about 0.1 picogram (pg) to about 100 pg, about 0.1 nanogram (ng) to about 100 ng, about 0.1 microgram (μg) to about 100 μg, about 0.1 milligram (mg) to about 100 mg; about 0.1 picomolar (pM) to about 100 pM, about 0.1 nanomolar (nM) to about 100 nM, about 0.1 micromolar (μM) to about 100 μM, about 0.1 millimolar (mM) to about 1 mM; or about 0.1 mg/kg to about 100 mg/kg.

In some embodiments, a compound disclosed herein is present in a pharmaceutical composition or unit dosage form in an amount of about 1 nmol, about 2 nmol, about 3 nmol, about 4 nmol, about 5 nmol, about 6 nmol, about 7 nmol, about 8 nmol, about 9 nmol, about 10 nmol, about 11 nmol, about 12 nmol, about 13 nmol, about 14 nmol, about 15 nmol, about 16 nmol, about 17 nmol, about 18 nmol, about 19 nmol, or about 20 nmol.

In some embodiments, a compound disclosed herein is present in a pharmaceutical composition or unit dosage form in an amount of about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, about 19 nM, or about 20 nM.

In some embodiments, a compound disclosed herein is administered to subject in a dose amount of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg.

Non-limiting examples of pharmaceutically-acceptable excipients suitable for use herein further include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, anti-microbial agents, plant cellulosic material and spheronization agents, and any combination thereof.

A composition of the present disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Disclosed methods include administration of a miRNA-221-5p mimic, or a pharmaceutically-acceptable salt thereof, in combination with a pharmaceutically-acceptable carrier. The carrier can be selected to minimize any degradation of the active ingredient and to minimize any adverse side effects in the subject.

Methods disclosed herein include administration of a compound that reduces, inhibits, or suppresses NLRP3 expression, or a pharmaceutically-acceptable salt thereof, in combination with a pharmaceutically-acceptable carrier. The carrier can be selected to minimize any degradation of the active ingredient and to minimize any adverse side effects in the subject.

A compound or a pharmaceutically-acceptable salt thereof herein can be conveniently formulated into pharmaceutical compositions composed of one or more pharmaceutically-acceptable carriers. See e.g., Remington's Pharmaceutical Sciences, latest edition, by E.W. Martin Mack Pub. Co., Easton, Pa., which discloses typical carriers, including fluid carriers, and conventional methods of preparing pharmaceutical compositions that can be used in conjunction with the preparation of formulations of the compound described herein and which is incorporated by reference herein. Such pharmaceuticals can be standard carriers for administration of compositions to humans and non-humans, including solutions such as sterile water, saline, and buffered solutions at physiological pH. Other compositions can be administered according to standard procedures. For example, pharmaceutical compositions can also include one or more additional active ingredients such as antimicrobial agents, anti-inflammatory agents, and anesthetics.

Non-limiting examples of pharmaceutically-acceptable liquid carriers include water, saline solution, Ringer's solution, glycerol, ethanol, and dextrose solution. The pH of the solution can be from about 5 to about 8, and can be from about 7 to about 7.5. Further carriers include sustained release preparations such as semipermeable matrices of solid hydrophobic polymers containing the compound or a pharmaceutically-acceptable salt thereof, where the matrices are in the form of shaped articles, such as films, liposomes, microparticles, and microcapsules.

Methods disclosed herein relate to administering the compound or a pharmaceutically-acceptable salt thereof as part of a pharmaceutical composition. The disclosed methods relate to administering the compound that suppresses NLRP3 expression or a pharmaceutically-acceptable salt thereof as part of a pharmaceutical composition. In various embodiments, compositions of the present disclosure can comprise a liquid comprising an active agent in solution, in suspension, or both. Liquid compositions can include gels. In some embodiments, the liquid composition is aqueous. Alternatively, the composition can take the form of an ointment. In some embodiments, the composition is an in situ gellable aqueous composition. In some embodiments, the composition is an in situ gellable aqueous solution.

Pharmaceutical compositions containing the compounds described herein can be administered for prophylactic or therapeutic treatments. Compositions can contain any number of active agents. In therapeutic applications, the compositions can be administered to a subject already suffering from a disease or condition, in an amount sufficient to cure or at least partially arrest the symptoms of the disease or condition, or to cure, heal, improve, reduce, lessen, ameliorate, or reduce a likelihood of occurrence of the disease or condition. Compounds can also be administered to lessen or reduce a likelihood of developing, contracting, or worsening a condition. Amounts effective for this use can vary based on the severity and course of the disease or condition, previous therapy, the subject's health status, weight, response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple injections. The compounds can be packed together or separately, in a single package or in a plurality of packages. One or all of the therapeutic agents can be given in multiple doses. If not simultaneous, then the timing between the multiple doses can vary.

The compounds described herein can be administered before, during, or after the occurrence of a disease or condition, and the timing of administering the composition containing a compound can vary. For example, the compounds can be used as a prophylactic and can be administered continuously to subjects with a propensity to conditions or diseases to lessen or reduce a likelihood of the occurrence of the disease or condition. The compounds and compositions can be administered to a subject during or as soon as possible after the onset of the symptoms. The administration of the compounds can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Compounds and compositions described herein can be packaged as a kit. In some embodiments, provided herein is a kit comprising a compound disclosed herein, or a pharmaceutically-acceptable salt thereof, and written instructions on use of the kit in the treatment of a condition described herein. In some embodiments, the present disclosure provides a kit comprising a compound disclosed herein, or a pharmaceutically-acceptable salt thereof, an antibody, and written instructions on use of the kit in the treatment of a condition described herein.

A sequence of a compound disclosed herein can have at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to human miRNA-221-5p (i.e., SEQ ID NO: 2).

A sequence of a compound herein can have at least about 70% homology, at least about 71% homology, at least about 72% homology, at least about 73% homology, at least about 74% homology, at least about 75% homology, at least about 76% homology, at least about 77% homology, at least about 78% homology, at least about 79% homology, at least about 80% homology, at least about 81% homology, at least about 82% homology, at least about 83% homology, at least about 84% homology, at least about 85% homology, at least about 86% homology, at least about 87% homology, at least about 88% homology, at least about 89% homology, at least about 90% homology, at least about 91% homology, at least about 92% homology, at least about 93% homology, at least about 94% homology, at least about 95% homology, at least about 96% homology, at least about 97% homology, at least about 98% homology, at least about 99% homology, at least about 99.1% homology, at least about 99.2% homology, at least about 99.3% homology, at least about 99.4% homology, at least about 99.5% homology, at least about 99.6% homology, at least about 99.7% homology, at least about 99.8% homology, at least about 99.9% homology, at least about 99.91% homology, at least about 99.92% homology, at least about 99.93% homology, at least about 99.94% homology, at least about 99.95% homology, at least about 99.96% homology, at least about 99.97% homology, at least about 99.98% homology, or at least about 99.99% homology to human miRNA-221-5p (i.e., SEQ ID NO: 2).

Various methods and software programs can be used to determine the identity or homology between two or sequences, such as NCBI BLAST, Clustal W, MAFFT, Clustal Omega, AlignMe, Praline, or another suitable method or algorithm.

In some embodiments, a compound disclosed herein is at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides in length.

### Treatment Methods

Provided herein are methods for treating a subject afflicted with a musculoskeletal or connective tissue condition with a therapeutically-effective amount of an inhibitor of NLRP3, IL-1β, and/or TNFα. The subject can be a human. Treatment can include treating a human in a clinical trial. A treatment can comprise administrating to a subject a therapy that suppresses expression of NLRP3.

Methods described herein include methods for the treatment of tendon damage, including damage resulting from tendon injury and tendinopathy. Treatment can include modulating or promoting healing of tendon, increasing development or proliferation of tenocytes, modulating or increase relative collagen composition or synthesis in tendon (e.g., collagen I or collagen III), modulating or improving biomechanical properties of tendon (e.g., improving or increasing the tensile strength of the tendon), and relieving or reducing pain or discomfort resulting from tendon injury and tendinopathy.

Non-limiting examples of possible subjects for administration include the following. Subjects can be humans, non-human primates such as chimpanzees, and other apes and monkey species; farm animals such as cattle, horses, sheep, goats, and swine; domestic animals such as rabbits, dogs, and cats; and laboratory animals including rats, mice, and guinea pigs. A subject can be of any age. Subjects can be, for example, elderly adults, adults, adolescents, pre-adolescents, children, toddlers, and infants.

Subjects described herein in need of treatment can include subjects having reduced expression of miR-122-5p and/or subjects having elevated expression of NLRP3, IL-1β, and/or TNFα, as compared to subjects not in need of treatment for a musculoskeletal or connective tissue condition.

Non-limiting examples of musculoskeletal or connective tissue conditions include tendinopathy, tendonitis/tendinitis, tenosynovitis, tendon injury, acute tendon injury, chronic tendon injury, a tendon condition, a cartilage injury, a ligament condition, a soft tissue condition, a joint condition, a muscle condition, a bone condition, osteoporosis, osteoarthritis, arthritis, rheumatoid arthritis, gout, fibromyalgia syndrome, systemic lupus erythematosus, Sjögren's syndrome, an autoimmune condition, and a non-autoimmune condition.

### Activity Assays

Histological assays, e.g., tendon histology assays, can be used to assess therapeutic effectiveness of a compound herein. For example, the Movin scoring system can be used to classify the histopathology of tendinopathy. Variables included in the Movin scale are: (1) fiber structure; (2) fiber arrangement; (3) rounding of the nuclei; (4) regional variations in cellularity; (5) increased vascularity; (6) decreased collagen stainability; (7) hyalinization; and (8) glycosaminoglycan (GAG) content. Each variable is scored between 0 and 3, with 0 being normal, 1 slightly abnormal, 2 abnormal, and 3 markedly abnormal. Hematoxylin and eosin (H&E) staining can be used to assess the first seven variables. Alcian blue staining can be used to assess GAG content. The total semiquantitative histologic score can vary between 0 (normal tendon) and 24 (the most severe abnormality detectable).

Biological activity of a compound described herein can also be assessed by measuring expression levels of the target mRNA and downstream effectors. For example, miR-221-5p activity can be evaluated by measuring expression of NLRP3, Col I, Col III, IL-1β, and/or TNFα.

Nociception (pain) and mechanical sensibility assays can be used to assess therapeutic effectiveness of a compound herein. For example, the Von Frey assay that uses small pieces of nylon rod (known as Von Frey hairs or filaments) to test a rodent's sensitivity to a mechanical stimulus. In this test, the animal stands on an elevated mesh platform, and the Von Frey hairs are inserted through the mesh to poke the animal's hind paw. The animal normally reacts by withdrawing or shaking of the paw. The force at which this response occurs is known as the paw withdrawal threshold (PWT). An increased PWT can indicate increased sensibility to the stimulus due to alleviation tendinopathy pain symptoms.

### EXAMPLES

### Example 1. Efficacy and Safety of miR-221-5p on Tendinopathy

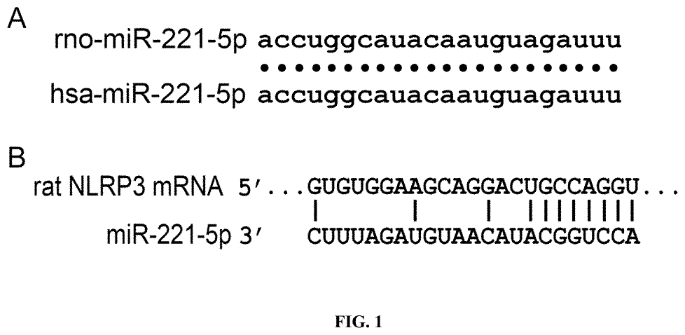

miR-221-5p is a small, noncoding RNA that is conserved between rat (rno-miR-221-5p; SEQ ID NO: 1) and human (hsa-miR-221-5p; SEQ ID NO: 2) as shown in FIG. 1, Panel A. miR-221-5p (SEQ ID NO: 4) inhibited NLRP3 expression via binding to rat NLRP3 3′-UTR (SEQ ID NO: 3) as shown in FIG. 1, Panel B.

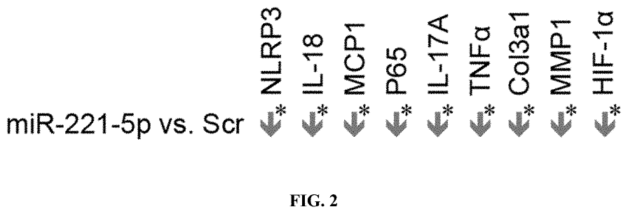

A miR-221-5p mimic (10 pM) suppressed levels of pro-inflammatory mediators and downstream factors, compared to that transfected with a scramble miRNA (Scr) in rat tenocytes in the presence of IL-1β for 24 hours as determined by real-time PCR (FIG. 2). These pro-inflammatory mediators and downstream factors include genes involved in the synthesis and degradation of ECM, for example, IL-18, MCP1, P65, IL-17A, TNFα, Col3a1, MMP1, and HIF-1a. Data were analyzed using GraphPad (*p<0.05 using Student's t-test).

Rats (male, 4-month old, Sprague Dawley) were subjected to collagenase-induced tendinopathy. The right calcaneal tendon (rat analogue of the human Achilles tendon) was injected with 10 μl collagenase (0.015 mg/ml) or 10 μl PBS as a sham control. One week after the collagenase injection, at a stage of mild tendinopathy, the tendinopathic tendons were further injected with: 1) miR-221-5p (20 pmol), or 2) scramble miRNA control (miR-Scr; 20 pmol) suspended in 10 μl PBS. The sham control animals were further injected with 10 μl PBS. Three weeks after this second injection, the rats were subjected to pain behavior testing using the Von Frey assay and then sacrificed. Whole blood was collected for complete blood count and serum chemistry analysis. The right hind limbs were fixed in formalin, the calcaneal tendon was dissected, and embedded in paraffin.

H&E analysis revealed that while the calcaneal tendons of sham animals exhibited histologic features seen in healthy tendons, tendinopathic rats treated with miR-Scr exhibited features typically observed in tendinopathy, including increased cellularity, a more rounded cell morphology, increased vascularity, and a disorganized arrangement of collagen fibers (FIG. 3, Panel A), thereby resulting in a higher Movin score (FIG. 3, Panel B). Rats treated with miR-Scr exhibited higher levels of cleaved type I collagen as assessed by immunohistochemistry staining using a primary antibody recognizing collagenase cleaved type I collagen (FIG. 3, Panels C and D). While type I collagen (Col I) expression was unchanged with either treatment (FIG. 3, Panel E), levels of type III collagen (Col III) were elevated with miR-Scr (FIG. 3, Panel F), thereby suggesting inferior tendon repair. The percentage of pro-inflammatory markers NLRP3 (FIG. 3, Panel G), IL-1β (FIG. 3, Panel H), and TNFα (FIG. 3, Panel I) were also increased in miR-Scr-treated tendons. In contrast, miR-221-5p mitigated the tendinopathic features that were observed (FIG. 3, Panels A-I). The Von Frey assay revealed that miR-Scr-treated rats exhibited a lower PWT, while miR-221-5p treatment showed a trend in alleviating this pain-related symptom (FIG. 3, Panel J). Complete blood counts and serum chemistry tests for liver and kidney function and electrolyte balance were not significantly different across all treatments, thereby suggesting no severe adverse effects with miR-221-5p. Data were analyzed using GraphPad (*p<0.05, **p<0.01 using one-way ANOVA.

### Example 2. Chemical Modifications of miR-221-5p

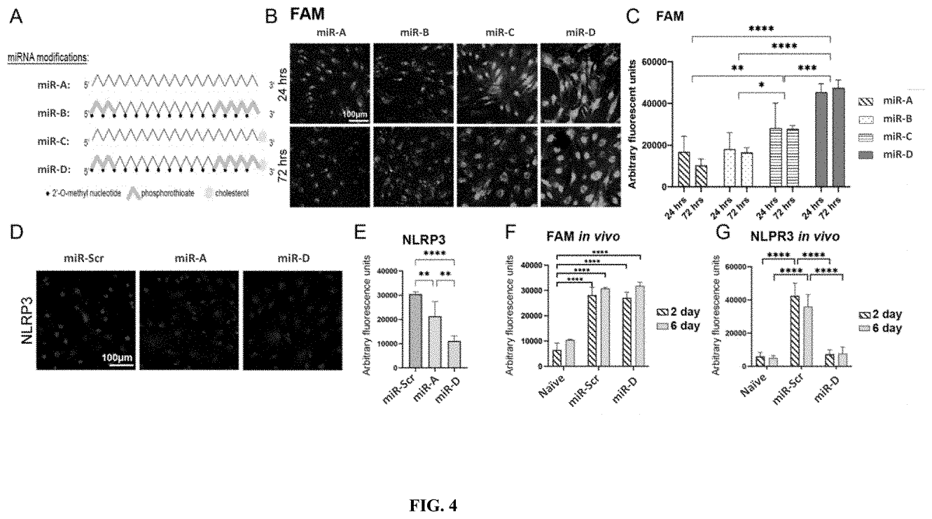

Chemical modifications of oligonucleotides can be used in oligonucleotide drug development to enhance stability and delivery to the target. Various double-stranded miR-221-5p mimics were designed and tested in transfection into human primary tenocytes to assess stability and uptake enhancement. The “guide” or “sense” strands were not modified to preserve the silencing activity of miR-221-5p, while the “passenger” or “antisense” strands were modified as shown in FIG. 4, Panel A. miR-A is an unmodified antisense strand of miR-221-5p. This antisense strand was modified to produce miR-B, miR-C, and miR-D. In miR-B, the antisense strand was modified to include 2′-O-methyl-modified nucleotides, and six of the backbone linkages were substituted with phosphorothioate bonds while the remaining linkages were phosphodiester bonds (two at the 5′-end and four at the 3′-end). In miR-C, cholesterol was conjugated to the 3′-end, while the bases remained unmodified. In miR-D, the double-stranded miR-221-5p was chemically modified as in miR-B to include 2′-O-methyl-modified nucleotides and six backbone linkages substituted with phosphorothioate bonds (two at the 5′-end and four at the 3′-end), and was also conjugated with cholesterol (dual modifications). miR-Scr (scramble miRNA) served as a control. To facilitate tracking of the miRNA, each of the above-described miRNA was conjugated with a fluorescent label (i.e., FAM) on the 3′-end.

The miR-221-5p mimics (20 nmol) were transfected in human primary tenocytes (87 year old male) or in rat Achilles tendon (20 pmol) using Lipofectamine RNAiMAX or in vivo-jetPEI (Polyplus transfection), respectively. Data were analyzed using GraphPad (*p<0.05, **p<0.01, ***p<0.001, ****p<0.0001 using Sidak's multiple comparison test in a mixed-effects model in FIG. 4, Panel C, one-way ANOVA in FIG. 4, Panel E, or two-way ANOVA in FIG. 4, Panels F and G).

Fluorescent imaging confirmed successful transfection of these miRNA into human primary tenocytes in the presence of IL-1β (FIG. 4, Panel B). The transfection rates after transfection for 24 and 72 hours determined based on cell count (% of FAM-positive cells divided by % of DAPI-positive cells) were 68% and 68% for miR-A, 72% and 77% for miR-B, 89% and 92% for miR-C, and 98% to 99% for miR-D, respectively. While miR-221-5p uptake did not significantly differ between 24 and 72 hours after transfection for all 4 mimics, miR-C and miR-D led to enhanced and sustained uptake of miR-221-5p at 24 and 72 hours after transfection compared to miR-A (FIG. 4, Panel C). miR-D exhibited a synergistic effect of increasing miR-221-5p uptake compared to all other mimics (FIG. 4, Panel C).

To assess whether the chemical modifications altered the silencing activity of miR-221-5p, expression of NLRP3, a target gene of miR-221-5p, was evaluated. Protein level of NLRP3 was significantly decreased at 72 hours after miR-221-5p treatment compared to after scramble treatment in human tenocytes, and miR-D treatment further reduced NLRP3 levels compared to miR-A treatment (FIG. 4, Panels D and E). The data suggest chemical modification of miR-221-5p does not alter the effect of miR-221-5p in suppressing the transcription of NLRP3, and the higher level of miR-D at 72 hours may account for the stronger biological effect in suppressing NLRP3 expression.

To confirm the activity of miR-221-5p in vivo, miR-D and miR-Scr were injected into the Achilles tendon of rats one week after collagenase-induced tendinopathy. The transfection rate at 2 and 6 days was 49% and 63% for miR-Scr, respectively, and 66% and 72% for miR-D, respectively. These results are consistent with transfection rates reported in the literature. Levels of both miR-Scr and miR-D as indicated by immunofluorescence staining for FAM were detected at significantly high levels at 2 days after miRNA injection. These levels were sustained until at least 6 days after injection, in contrast to that in the contralateral tendon which was used as a naïve control (FIG. 4, Panel F). The injection of miR-D, but not miR-Scr, led to a decrease in NLRP3 expression in vivo at both 2 and 6 days after injection (FIG. 4, Panel G).

### EMBODIMENTS

Embodiment 1. A pharmaceutical composition comprising in a unit dosage form: a therapeutically-effective amount of a compound that increases activity of miRNA-221-5p; and a pharmaceutically-acceptable carrier.

Embodiment 2. The pharmaceutical composition of embodiment 1, wherein the compound is a microRNA or mimic thereof.

Embodiment 3. The pharmaceutical composition of embodiment 2, wherein the compound is conjugated to a serum lipoprotein.

Embodiment 4. The pharmaceutical composition of embodiment 3, wherein the serum lipoprotein is cholesterol.

Embodiment 5. The pharmaceutical composition of any one of embodiments 2-4, wherein the compound comprises a ribose modification.

Embodiment 6. The pharmaceutical composition of embodiment 5, wherein the ribose modification is a 2′-O-methyl modification.

Embodiment 7. The pharmaceutical composition of any one of embodiments 2-6, wherein the compound comprises a phosphate backbone modification.

Embodiment 8. The pharmaceutical composition of embodiment 7, wherein the phosphate backbone modification is a phosphorothioate modification.

Embodiment 9. The pharmaceutical composition of embodiment 1, wherein the compound is a precursor of a microRNA or a mimic thereof.

Embodiment 10. The pharmaceutical composition of embodiment 1, wherein the compound encodes a microRNA or a mimic thereof.

Embodiment 11. The pharmaceutical composition of embodiment 1, wherein the compound encodes a precursor of a microRNA or a mimic thereof.

Embodiment 12. The pharmaceutical composition of any one of embodiments 2-11, wherein the microRNA is miRNA-221-5p.

Embodiment 13. The pharmaceutical composition of any one of embodiments 1-12, wherein the compound reduces NLRP3 expression.

Embodiment 14. The pharmaceutical composition of any one of embodiments 1-13, wherein the compound binds to NLRP3 mRNA.

Embodiment 15. The pharmaceutical composition of any one of embodiments 1-14, wherein the compound binds to a 3′-UTR of NLRP3 mRNA.

Embodiment 16. The pharmaceutical composition of any one of embodiments 1-15, wherein the compound is encapsulated in a microvesicle.

Embodiment 17. The pharmaceutical composition of any one of embodiments 1-16, wherein the pharmaceutical composition is formulated for injection.

Embodiment 18. The pharmaceutical composition of any one of embodiments 1-17, wherein the pharmaceutical composition is formulated for intratendinous injection.

Embodiment 19. A method of treating a connective tissue condition in a subject in need thereof, comprising administering to the subject a therapeutically-effective amount of a compound that increases activity of miRNA-221-5p.

Embodiment 20. The method of embodiment 19, wherein the compound is a microRNA or mimic thereof.

Embodiment 21. The method of embodiment 20, wherein the compound is conjugated to a serum lipoprotein.

Embodiment 22. The method of embodiment 21, wherein the serum lipoprotein is cholesterol.

Embodiment 23. The method of any one of embodiments 20-23, wherein the compound comprises a ribose modification.

Embodiment 24. The method of embodiment 23, wherein the ribose modification is 2′-O-methyl modification.

Embodiment 25. The method of any one of embodiments 20-24, wherein the compound comprises a phosphate backbone modification.

Embodiment 26. The method of embodiment 25, wherein the phosphate backbone modification is a phosphorothioate modification.

Embodiment 27. The method of embodiment 19, wherein the compound is a precursor of a microRNA or a mimic thereof.

Embodiment 28. The method of embodiment 19, wherein the compound encodes a microRNA or a mimic thereof.

Embodiment 29. The method of embodiment 19, wherein the compound encodes a precursor of a microRNA or a mimic thereof.

Embodiment 30. The method of any one of embodiments 20-29, wherein the microRNA is miRNA-221-5p.

Embodiment 31. The method of any one of embodiments 19-30, wherein the compound reduces NLRP3 expression.

Embodiment 32. The method of any one of embodiments 19-31, wherein the compound binds to NLRP3 mRNA.

Embodiment 33. The method of any one of embodiments 19-32, wherein the compound binds to a 3′-UTR of NLRP3 mRNA.

Embodiment 34. The method of any one of embodiments 19-33, wherein the compound is encapsulated in a microvesicle.

Embodiment 35. The method of any one of embodiments 19-34, wherein the compound is formulated in a pharmaceutical composition that comprises a pharmaceutically-acceptable carrier.

Embodiment 36. The method of any one of embodiments 19-35, wherein the administering is by injection.

Embodiment 37. The method of any one of embodiments 19-36, wherein the administering is by intratendinous injection.

Embodiment 38. The method of any one of embodiments 19-37, wherein the connective tissue condition is tendinopathy.

Embodiment 39. The method of any one of embodiments 19-38, wherein the connective tissue condition is tendonitis.

Embodiment 40. The method of any one of embodiments 19-39, wherein the connective tissue condition is a tendon injury.

Embodiment 41. The method of any one of embodiments 19-40, further comprising assaying the subject for modulation of gene expression of NLRP3.

Embodiment 42. The method of any one of embodiments 19-41, further comprising assaying the subject for modulation of gene expression of IL-1β.

Embodiment 43. The method of any one of embodiments 19-42, further comprising assaying the subject for modulation of gene expression of TNFα.

Embodiment 44. The method of any one of embodiments 19-43, further comprising assaying the subject for modulation of gene expression of type I collagen.

Embodiment 45. The method of any one of embodiments 19-44, further comprising assaying the subject for modulation of gene expression of type III collagen.

Embodiment 46. The method of any one of embodiments 19-45, further comprising assaying the subject for modulation of a ratio of gene expression of type I collagen to gene expression of type III collagen.

Embodiment 47. A method of treating a musculoskeletal condition in a subject in need thereof, comprising administering to the subject a therapeutically-effective amount of a compound that increases activity of miRNA-221-5p.

Embodiment 48. The method of embodiment 47, wherein the compound is a microRNA or mimic thereof.

Embodiment 49. The method of embodiment 48, wherein the compound is conjugated to a serum lipoprotein.

Embodiment 50. The method of embodiment 49, wherein the serum lipoprotein is cholesterol.

Embodiment 51. The method of any one of embodiments 48-50, wherein the compound comprises a ribose modification.

Embodiment 52. The method of embodiment 51, wherein the ribose modification is a 2′-O-methyl modification.

Embodiment 53. The method of any one of embodiments 48-52, wherein the compound comprises a phosphate backbone modification.

Embodiment 54. The method of embodiment 53, wherein the phosphate backbone modification is a phosphorothioate modification.

Embodiment 55. The method of embodiment 47, wherein the compound is a precursor of a microRNA or a mimic thereof.

Embodiment 56. The method of embodiment 47, wherein the compound encodes a microRNA or a mimic thereof.

Embodiment 57. The method of embodiment 47, wherein the compound encodes a precursor of a microRNA or a mimic thereof.

Embodiment 58. The method of any one of embodiments 48-57, wherein the microRNA is miRNA-221-5p.

Embodiment 59. The method of any one of embodiments 47-58, wherein the compound reduces NLRP3 expression.

Embodiment 60. The method of any one of embodiments 47-59, wherein the compound binds to NLRP3 mRNA.

Embodiment 61. The method of any one of embodiments 47-60, wherein the compound binds to a 3′-UTR of NLRP3 mRNA.

Embodiment 62. The method of any one of embodiments 47-61, wherein the compound is encapsulated in a microvesicle.

Embodiment 63. The method of any one of embodiments 47-62, wherein the compound is formulated in a pharmaceutical composition that comprises a pharmaceutically-acceptable carrier.

Embodiment 64. The method of any one of embodiments 47-63, wherein the administering is by injection.

Embodiment 65. The method of any one of embodiments 47-64, wherein the administering is by intraarticular injection.

Embodiment 66. The method of any one of embodiments 47-65, wherein the administering is by intramuscular injection.

Embodiment 67. The method of any one of embodiments 47-66, wherein the musculoskeletal condition is arthritis.

Embodiment 68. The method of any one of embodiments 47-67, wherein the musculoskeletal condition is rheumatoid arthritis.

Embodiment 69. The method of any one of embodiments 47-68, wherein the musculoskeletal condition is osteoarthritis.

Embodiment 70. The method of any one of embodiments 47-69, further comprising assaying the subject for modulation of gene expression of NLRP3.

Embodiment 71. The method of any one of embodiments 47-70, further comprising assaying the subject for modulation of gene expression of IL-1β.

Embodiment 72. The method of any one of embodiments 47-71, further comprising assaying the subject for modulation of gene expression of TNFα.

Embodiment 73. The method of any one of embodiments 47-72, further comprising assaying the subject for modulation of gene expression of type I collagen.

Embodiment 74. The method of any one of embodiments 47-73, further comprising assaying the subject for modulation of gene expression of type III collagen.

Embodiment 75. The method of any one of embodiments 47-74, further comprising assaying the subject for modulation of a ratio of gene expression of type I collagen to gene expression of type III collagen.

1. A pharmaceutical composition comprising in a unit dosage form: a therapeutically-effective amount of a compound that increases activity of miRNA-221-5p; and a pharmaceutically-acceptable carrier.

2. The pharmaceutical composition of claim 1, wherein the compound is a microRNA or mimic thereof.

3. The pharmaceutical composition of claim 2, wherein the compound is conjugated to a serum lipoprotein.

4. The pharmaceutical composition of claim 3, wherein the serum lipoprotein is cholesterol.

5. The pharmaceutical composition of claim 2, wherein the compound comprises a ribose modification.

6. The pharmaceutical composition of claim 5, wherein the ribose modification is a 2′-O-methyl modification.

7. The pharmaceutical composition of claim 2, wherein the compound comprises a phosphate backbone modification.

8. The pharmaceutical composition of claim 7, wherein the phosphate backbone modification is a phosphorothioate modification.

9. The pharmaceutical composition of claim 2, wherein the microRNA is miRNA-221-5p.

10. The pharmaceutical composition of claim 1, wherein the compound is a precursor of a microRNA or a mimic thereof.

11. The pharmaceutical composition of claim 1, wherein the compound encodes a microRNA or a mimic thereof.

12. The pharmaceutical composition of claim 1, wherein the compound encodes a precursor of a microRNA or a mimic thereof.

13. The pharmaceutical composition of claim 1, wherein the compound reduces NLRP3 expression.

14. The pharmaceutical composition of claim 1, wherein the compound binds to NLRP3 mRNA.

15. The pharmaceutical composition of claim 1, wherein the compound binds to a 3′-UTR of NLRP3 mRNA.

16. The pharmaceutical composition of claim 1, wherein the compound is encapsulated in a microvesicle.

17. The pharmaceutical composition of claim 1, wherein the pharmaceutical composition is formulated for injection.

18. The pharmaceutical composition of claim 1, wherein the pharmaceutical composition is formulated for intratendinous injection.

19-75. (canceled)

76. The pharmaceutical composition of claim 1, wherein the compound reduces IL-1β expression.

77. The pharmaceutical composition of claim 1, wherein the compound reduces TNFα expression.

78. The pharmaceutical composition of claim 1, wherein the compound reduces type I collagen expression.

79. The pharmaceutical composition of claim 1, wherein the compound reduces type III collagen expression.


SEQUENCE LISTING (4 sequences)

SEQ ID NO: 1 - RNA, 22 nt, Rattus sp.
  source 1..22: mol_type = genomic RNA
acctggcatacaatgtagattt

SEQ ID NO: 2 - RNA, 22 nt, Homo sapiens
  source 1..22: mol_type = genomic RNA
acctggcatacaatgtagattt

SEQ ID NO: 3 - RNA, 23 nt, Rattus sp.
  source 1..23: mol_type = genomic RNA
gtgtggaagcaggactgccaggt

SEQ ID NO: 4 - RNA, 23 nt, unidentified
  source 1..23: mol_type = unassigned RNA
acctggcatacaatgtagatttc